(19) United States
(12) Patent Application Publication
(10) Pub. No.: US 2011/0257546 A1
(43) Pub. Date: Oct. 20, 2011

(54) Biometric Sensor And Heart Function Monitoring Apparatus

(75) Inventors: Giovanni Gozzini, Berkeley, CA (US); Michele Borgatti, Bologna (IT)

(73) Assignee: UPEK, INC., Emeryville, CA (US)

(21) Appl. No.: 12/763,135

(22) Filed: Apr. 19, 2010

(51) Int. Cl. A61B 5/0404; A61B 5/117; G06K 9/00

(52) U.S. Cl. 600/509; 382/124

(57) ABSTRACT

A heartbeat monitoring circuit shares one electrode with another device, such as a biometric sensor, and a second electrode that may be a dedicated element or form a part of the case of the apparatus in which the circuit is housed. When operated as an electrode for the heartbeat monitoring circuit, the biometric sensor is contacted by the user's finger while the second electrode is contacted by another part of the user's body, such as a finger on the hand holding the apparatus (e.g., cell phone). Contact may then simply and naturally be made on either side of the user's heart, and ideal arrangement for parametric heart data acquisition. A third electrode may optionally be provided. Data analysis may be performed on the apparatus or by a remote optional processing center.

### BACKGROUND

The present disclosure is related to devices for collecting and determining parametric data related to a user's heart function, and more specifically to an electrode arrangement therefor which utilizes a biometric sensor as one electrode in a multi-electrode data gathering circuit.

The present disclosure brings together several different technologies. First are methods and devices for measuring electrical cell activity. One specific example of such electrical cell activity is the electrical waveform which drives a heartbeat. While devices used in the field of electrocardiography (ECG or EKG) measure the electrical activity of cells related to heart activity, many other similar measurements are employed in other field such as electroencephalography (EEG) for brain activity, electromyography (EMG) for muscle activity, etc. For the purposes hereof, we focus on devices for collecting and determining parametric data related to heart functions, although the background and embodiments described herein are equally applicable to cell activity monitoring in other fields.

Heartbeat measurement and heart monitor devices are well known and widely available today in many different forms and with a range of functions and processing. Heart monitor devices range from relatively large free-standing devices to very compact devices wearable by a user. While simple devices measure only a heartbeat rate, more sophisticated devices measure many aspects of heart health and operation. Devices for such measurement may be dedicated to monitoring, or the heart monitor functionality may be integrated into an apparatus serving other functions, such as a timepiece (e.g., U.S. Pat. No. 7,654,732), eyewear (e.g., U.S. Pat. No. 6,431,705), integrated into garments (e.g., U.S. Pat. No. 6,930,608), the handlebars of a bicycle (e.g., U.S. Pat. No. 4,319,581), etc.

While it is possible to measure heart beat rate optically, and/or with a single sensor, examination of the details of the heart pulse, for example the length, strength, etc. of the various stages of a heart pulse, is accomplished electrically and with data provided by a plurality of electrodes arranged at various locations on a user, and on opposite sides of the heart. Such measurement is referred to as diametric measurement since there are generally at least two electrodes located across the heart (e.g., left and rights sides) from one another. A reference voltage is determined, and then voltage variation is measured across the heart as it pumps. Electrical heart performance parameters, such as the length and shape of electrical waves that cause a heart to beat, may then be determined.

The second technology of relevance here are methods and devices for user identification and identity verification employing a biometric attribute of a user such as a fingerprint. For the purposes hereof, we focus on fingerprint sensors within the class of biometric sensors, although the background and embodiments described herein are equally applicable to sensors for other biometric attributes such as retinal scanning, voice recognition, and other attributes of a body of a user, as well as combinations thereof.

Fingerprint sensors and associated verification functionality are well known. Devices designed for sensing the pattern of a fingerprint fall into several categories based on the type of sensor they employ, such as optical, thermal, capacitive, and so on. In many such devices, the sensed portion of a user (e.g., the fingertip) must be essentially touching a portion of the sensor and a bezel associated therewith. The bezel may either place a charge on the finger or establish a fixed finger potential, which is used to enhance the sensing of the ridges and valleys of the fingerprint as they affect the electric field between the finger and the fingerprint sensing element plates and/or may be used for finger detection, for example to initiate a fingerprint scan. See, for example, U.S. Pat. No. 6,512,381.

Due to small size, low power consumption, ease of integration, etc., fingerprint sensors such as described above are often used in portable electronic devices, such as cell phones, PDAs, portable computers (e.g., notebook and netbook), etc. As the power, memory, capability, and ultimately populations of these devices increase, more and more sensitive, private data is being stored on them. In order to prevent unwanted use of the device or the data stored there in the event of loss or theft, such devices are often provided with fingerprint sensors such that the devices may only be used if the user's fingerprint matches a pre-enrolled fingerprint.

Certain types of fingerprint sensors include the ability to detect a user's pulse. For example, U.S. Pat. No. 7,254,255 teaches an optical fingerprint scanner which includes an infrared (IR) source and sensor. Associated with the optical sensing of the user's fingerprint, the IR source is applied to the fingertip. IR light transmitted by the fingertip is detected by the sensor and analyzed. The IR transmission characteristics of a living body are different than those for a non-living body, based on blood flow through the fingertip. Based on this difference, it can be determined from the detected transmission whether the fingertip being scanned is part of a living body, or whether is it not, in which case there is a high probability that the user is attempting to circumvent (or “spoof”) the fingerprint sensor.

However, there currently is no device that takes advantage of the physical placement of a fingertip on a fingerprint sensor and the placement of another portion of the user's body (e.g., fingertip of the other hand) on another portion of a handheld device to obtain electrical heart performance data.

### SUMMARY

Accordingly, the present disclosure is directed to an electrical cell function (e.g., heartbeat) monitoring circuit that shares one electrode with another device, such as a biometric (e.g., fingerprint) sensor. While often integrated into a hand-held device, the invention disclosed herein may form a part of any apparatus in which a biometric sensor is incorporated and in which at least one additional electrode is provided.

According to one aspect of the disclosure a mobile telephone or similar hand-held apparatus of a type that includes a fingerprint sensor is provided with a first electrode that may be conveniently contacted by a user as the apparatus is being used. The fingerprint sensor includes a conductive element such as a bezel or the like which forms a part of the sensor or is otherwise associated therewith. The apparatus includes circuitry for switching the function of the conductive element portion of the fingerprint sensor from a first state operable as a part of the fingerprint sensor circuit to a second state operable as an electrode for a parametric heart data monitor circuit.

According to another aspect of the present disclosure, a cell phone, PDA, portable music player (e.g., iPods, MP3 player), portable computer (e.g., notebook and netbook), tablet computer, remote control device, etc. is provided with at least two electrodes and circuitry for both a fingerprint sensing functionality and a parametric heart data monitor functionality. One of the at least two electrodes may alternatively form a part of the fingerprint sensing circuitry or the parametric heart data monitor circuitry. In variations of this aspect, the one electrode may simultaneously operate as a part of the fingerprint sensing circuitry and the parametric heart data monitor circuitry.

According to another aspect of the present disclosure, the first electrode is sized and positioned such that when the apparatus is held in a user's first hand a portion of that hand is in physical and electrical contact with the first electrode. In operation, a finger of the user's second hand contacts the conductive element of the fingerprint sensor. In a mode in which the conductive element of the fingerprint sensor is operable as an electrode for the parametric heart data monitor circuit, a connection is established between first and second hands across the user's body, permitting the diametric sensing of parametric heart data.

According to yet another aspect of the disclosure, the biometric sensor is a capacitive fingerprint sensor, and the apparatus includes circuitry that in a first mode of operation is operable to detect and digitize a fingerprint. The circuitry is further operable in a second mode of operation to detect and digitize parametric heart data of a user by employing the first electrode and the conductive element of the biometric sensor as a second electrode.

According to a still further aspect of the present disclosure, the dual functionality of the biometric sensor may be integrated into a device not traditionally held in the hand of a user during use, such as door locks, keyboards of desktop computers, and so forth. The body of the apparatus into which the biometric sensor is integrated is provided with an electrode that operates together with the biometric sensor to provide two electrodes for a parametric heart data monitor circuit.

According to yet another aspect of the present disclosure, a second electrode is provided in, on or communicatively coupled with an apparatus incorporating a biometric sensor. The second electrode, together with the first electrode and the conductive element of the biometric sensor, thereby provide a three-electrode arrangement for a parametric heart data monitor circuit.

A number of benefits are realized by utilizing the conductive portion associated with the fingerprint sensor as an electrode for parametric heart data acquisition. For example, system cost is reduced (e.g., the sensor internal logic can be used to sense and digitize both fingerprint data and parametric heart data). As another example, convenience is enhanced (e.g., the same finger position on the sensor can be scanned for fingerprint and used as one contact for the parametric heart data. As a further example, security is enhanced (e.g., the fingerprint and parametric heart data are sensed from the same finger at nearly the same time, adding a level of certainty that the parametric heart data originates with the person identified by the fingerprint authentication).

The above is a summary of a number of the unique aspects, features, and advantages of the present disclosure. However, this summary is not exhaustive. Thus, these and other aspects, features, and advantages of the present disclosure will become more apparent from the following detailed description and the appended drawings, when considered in light of the claims provided herein.

### BRIEF DESCRIPTION OF THE DRAWINGS

In the drawings appended hereto like reference numerals denote like elements between the various drawings. While illustrative, the drawings are not drawn to scale. In the drawings:

FIG. 1 is a perspective view of a mobile telephone incorporating a biometric sensor that provides both fingerprint sensing and an electrode for a parametric heart data monitor circuit according to an embodiment of the present disclosure.

FIG. 2 is a perspective view of the apparatus of FIG. 1 shown held in a user's hand in one mode of operation according to an embodiment of the present disclosure.

FIG. 3 is a top plan view of a biometric sensor with bezel structure that may be employed in apparatus according to an embodiment of the present disclosure.

FIG. 4 is a functional diagram of a circuit in which a biometric sensor circuit and parametric heart data monitor circuit are enabled by a switched connection through a biometric sensor according to an embodiment of the present disclosure.

FIG. 5A is a schematic diagram illustrating a 2-electrode circuit, and FIG. 5B is a 3-electrode circuit, for parametric heart data monitoring according to an embodiment of the present disclosure.

FIG. 6 is top plan view of a keyboard including a combined sensor and additional electrode according to an embodiment of the present disclosure.

FIG. 7 is an illustration of a three-electrode arrangement of a device incorporating a biometric sensor that provides both fingerprint sensing and an electrode for a parametric heart data monitor circuit according to an embodiment of the present disclosure.

### DETAILED DESCRIPTION

We initially point out that descriptions of well-known starting materials, processing techniques, components, equipment and other well known details are merely summarized or are omitted so as not to unnecessarily obscure the details of the present invention. Thus, where details are otherwise well known, we leave it to the application of the present invention to suggest or dictate choices relating to those details.

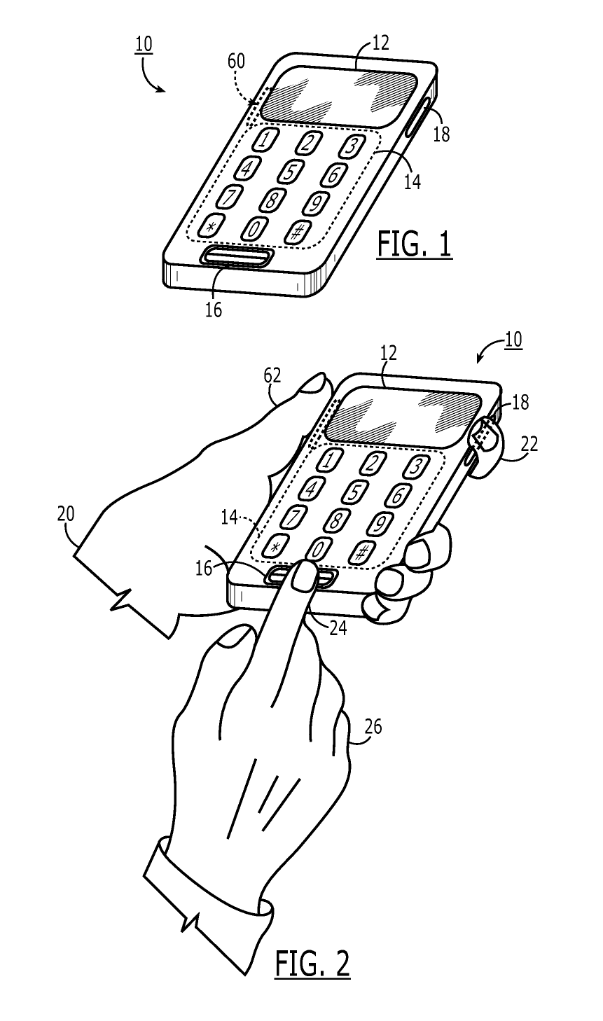

A first embodiment of a hand-held device that can operate both as a biometric sensor and electrical cell function (e.g., parametric heart data) monitoring apparatus 10 is shown in perspective view in FIG. 1. While the present disclosure may be useful for a variety of types of electrical cell function monitoring, to aid in clarity we focus here on heartbeat monitoring and heart function determination based on the monitored heartbeat. It will be appreciated, however, that this is simply one example of electrical cell function monitoring and that the present disclosure and claims appended hereto are not limited thereto.

In the embodiment of FIG. 1, the apparatus is a cellular telephone. It will be appreciated that other forms of handheld devices may equivalently embody the features described herein. Examples of such hand-held devices include cell phones, PDAs, portable music players (e.g., iPods, MP3 player), portable computers (e.g., notebook and netbook), tablet computers, remote control devices, etc. Furthermore, this disclosure and claims appended hereto may also find application to non-hand-held devices, and according the claims shall not be interpreted as being limited to hand-held devices except as they may specify any such limitation.

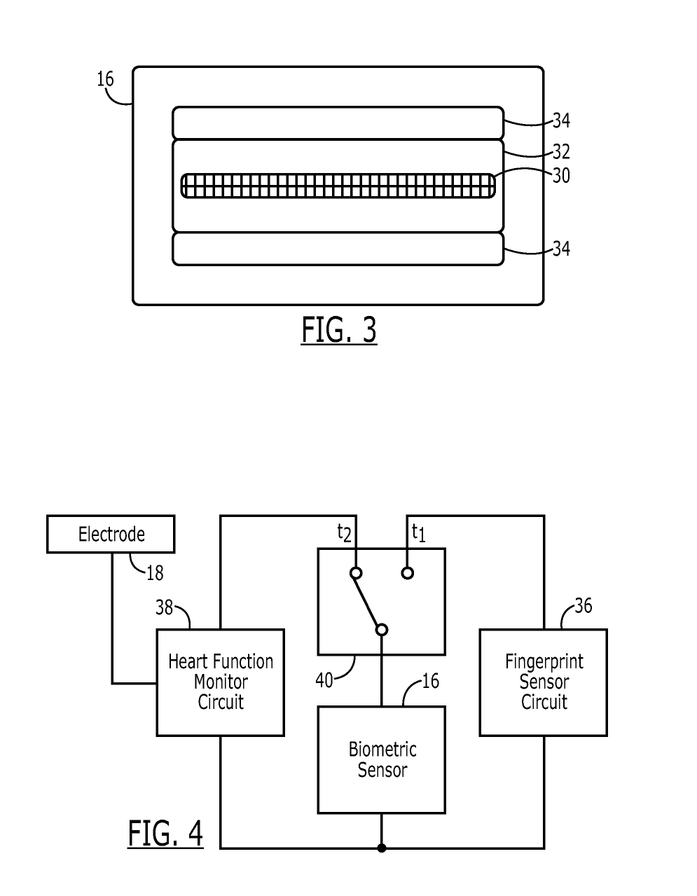

Apparatus 10 includes a display screen 12, keypad 14, and a fingerprint sensor 16. The fingerprint sensor 16 is of a type that includes a conductive region, such as a metal plate or bezel, sized and positioned for contact with a portion of a user's fingertip. Fingerprint sensor 16 may be of a type that senses an entire fingerprint as the fingertip rests still against the sensor (a so-called area sensor), or may be of a type in which small portions of a finger are scanned as the finger slides over the sensor, then reconstructs the full fingerprint in software (a so-called strip sensor). Fingerprint sensor 16 is described in more detail with reference to FIG. 3, below.

In the embodiment of FIG. 1, a first electrode 18 is provided on a side edge of apparatus 10. First electrode 18 is sized and positioned such that when a user holds the apparatus, at least a portion of one hand of a user may be made to comfortably and conveniently physically contact first electrode 18.

With reference to FIG. 2, apparatus 10 is of a size and shape such that it is relatively easily held in one hand 20 of a user. Ideally, first electrode 18 is sized and positioned such that when apparatus 10 is cradled in the palm of a user's hand 20, a portion of hand 20 such as finger 22 may make physical contact therewith. In particular, when a user holds apparatus 10 to allow for the application of the fingertip 24 of one hand 26 to fingerprint sensor 16, apparatus 10 is held in the other hand 20 such that fingertip 22 of that other hand 20 is in contact with first electrode 18. In this way, a user's left and right hands contact apparatus 10 simultaneously. Fingertip 22 is in contact with first electrode 18, and fingertip 24 is in contact with a portion of fingerprint sensor 16 that forms a second electrode in this mode of operation. Thus, contact with first and second electrodes on opposite sides of the body permits the diametric sensing of heart function data.

According to the above, at least a portion of fingerprint sensor 16 serves as a second electrode for the diametric sensing of heart function data. One embodiment of a fingerprint sensor 16 serving this function is illustrated in FIG. 3. Fingerprint sensor 16 consists of a sensor array 30 and related circuitry formed on surface of a semiconductor body, or die 32. Sensor array 30 is typically exposed for physical contact with a user's finger, or at most thinly covered with a protective material. Typically, the sensors operate according to principles that use the effect of various portions of a user's finger on a capacitive fringing field to construct an image of the user's fingerprint (generally referred to as a capacitive sensor). Accurate operation of such sensors can accommodate no more than a minimal gap between the sensor surface and the fingerprint to be sensed. Other types of sensors may also be employed, and the specific type of sensor shall not form a limitation of this disclosure or the claims unless specifically indicated as such.

A number of fingerprint sensor circuit designs operate by injecting a small current into the finger being sensed. One example of such a circuit is disclosed in U.S. Pat. No. 6,512,381, which is incorporated herein by reference. In order to drive the user's finger with the desired current, a contact structure, for example as disclosed in U.S. Pat. No. 6,636,053, which is also incorporated herein by reference, may be provided. The contact structure may take the form of a bezel 34 located near an edge of die 32, and often at both the front and rear edges of die 32. Bezel 34 has a generally planar upper surface that is either coplanar with or parallel to the plane of the upper surface of die 32. As the user applies a fingertip to the surface of the die, for example by placement on an area sensor or in the swiping motion over a strip sensor, the fingertip is simultaneously in physical and electrical contact with the surface of die 32 (i.e., sensor array 30 formed on the top surface of die 32) and bezel 34, the latter to electrically drive the fingertip during the sensing process. Other examples of conductive regions that may serve the purpose of driving the user's fingertip include a conductive structure or array of structures formed on or in die 32, a conductive bump or series of bumps formed separate from die 32 but positioned proximate thereto in the complete fingerprint sensor, bezel structures of shapes and locations other than as shown in FIG. 3, and so forth. While formed either separate from or as a part of die 32, the conductive regions form a part of the completed fingerprint sensor 16, and thus references herein to fingerprint sensor includes as an element thereof those conductive regions whatever their form and/or location unless otherwise specified.

FIG. 4 is a functional diagram illustrating the dual functionality provided by the fingerprint sensor 16 according to the present disclosure. In the embodiment illustrated in FIG. 4, the electrical driving of the fingertip is timed such that at time t₁ a current, voltage or charge is injected into the fingertip for the sensing function, and fingerprint sensor 16 operates as a fingerprint sensor for fingerprint sensing circuit 36. At a time t₂ the current for the fingerprint sensing function is off, and the circuit is connected for heart function monitoring. Fingerprint sensor 16 (again, at least the conductive regions thereof such as bezel 34, FIG. 3) then functions as one electrode for heart function monitor circuit 38. Switching between these functions may be controlled by a physical switching circuit 40, by software, or other methods and devices well known in the art.

As discussed further herein, the switched connections for the conductive portion of fingerprint sensor 16 may be made to accommodate one of three possible cases: (1) circuitry for heart function monitoring and fingerprint sensing are separate, sharing only the conductive portion of sensor 16 (and perhaps other system-level functions such as power supply, display drivers, and so forth), (2) circuitry for heart function monitoring and fingerprint sensing share modules such that certain elements providing heart function monitoring may be used to provide fingerprint sensing, and vice-versa, and (3) circuitry for heart function monitoring and fingerprint sensing are in fact part of the same circuitry.

In many embodiments, the conductive portion of fingerprint sensor 16 will serve the dual role of electrode for parametric heart data acquisition and as a portion of a fingerprint sensing circuit. However, in certain embodiment, the conductive portion associated with the fingerprint sensor may primarily or even strictly serve as an electrode for parametric heart data acquisition. Many of the benefits described above still apply, such as reduced cost and improved convenience and security.

Switching between heart function monitoring and fingerprint sensing may be accomplished in a variety of ways. First, when sensing a fingerprint is initiated (e.g., when a user attempts to access apparatus 10 into which fingerprint sensor 16 is incorporated) a clocking signal may be used to automatically periodically switch between functions performed by fingerprint sensor 16. Switching in this way as part of the determining access rights means that the heart function monitoring may provide an anti-spoofing function, ensuring that the proffered fingertip is living tissue and not a fraudulent facsimile nor disembodied digit. This anti-spoofing function may be separate from or form a part of heart function monitoring. Second, at any time that heart function monitoring is desired while access to apparatus 10 has been granted, the user may activate a heart function monitoring capability (e.g., running an application on apparatus 10). The function of fingerprint sensor 16 is then switched to operate as one electrode in a heart function monitoring circuit. Other techniques and timing for switching the function of fingerprint sensor 16 between fingerprint sensing and heart function monitoring may be employed without departing from the spirit and scope of the present disclosure and claims appended hereto.

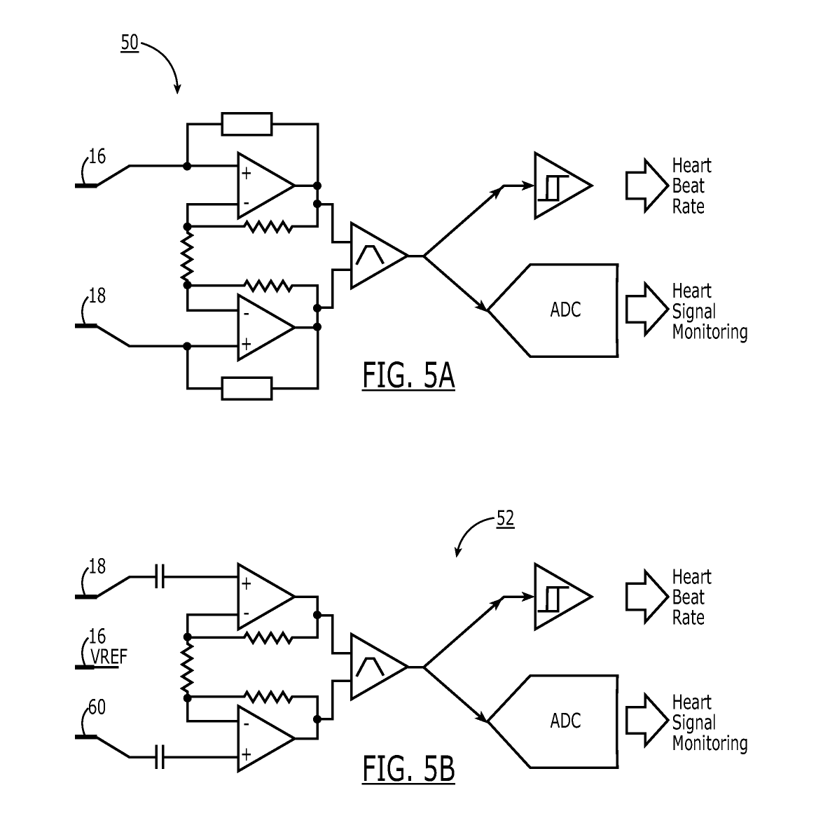

One exemplary circuit 50 employing biometric first electrode 18 and fingerprint sensor 16 as a second electrode for sensing and digitizing heart function data is shown in FIG. 5A. It will be understood that this circuit may be provided by way of a standalone component within apparatus 10 or integrated into other hardware such as the circuitry providing fingerprint capture and recognition. Furthermore, while sensing and digitizing are provided by circuit 50, additional processing may also be provided, such as display of heart function data on display 12, analysis of heart function data for selected responses, historical recording of heart functions, and so forth. Additional processing of digitized heart function data may also be performed by other elements resident on apparatus 10, such as one or more applications that may be run by apparatus 10, and by elements external to apparatus 10 operating on digitized heart function data transmitted thereto for example by wireless transmission, provided during a synchronization operation of apparatus 10, etc.

There are a variety of techniques that may be employed to sense a heartbeat useful with an arrangement of the type disclosed herein. For example, heartbeat detection may be accomplished by sensing the electrical signal that the skin of the user carries in different locations by virtue simply of the heartbeat itself. In this case, no signals need be injected into the skin. A simple comparator circuit for comparing the signal sensed at the various electrodes at fixed intervals of time may accomplish this function, as will be understood by one skilled in the art.

Returning to FIGS. 1 and 2, the description above has focused on two electrodes positioned for diametric sensing. However, a three-electrode circuit may be employed by adding a second electrode 60 (shown as hidden in FIG. 1 due to perspective) for example to the case of apparatus 10 opposite first electrode 18. When apparatus 10 is cradled in the palm of user's hand 20, a portion of hand 20 such as thumb 62 may make physical contact second electrode 60. In this way, two points of contact with a user's hand, at first and second electrodes 18 and 60, respectively, as well as a third point of contact at fingerprint sensor 16 are made, thus with left and right hands contacting apparatus 10 simultaneously. Contact with electrodes on opposite sides of the body thereby permits the diametric sensing of a heartbeat, with the three electrodes providing heartbeat monitoring capabilities in addition to those provided by a two-electrode arrangement, as well understood by one skilled in the art. An example of a 3-electrode circuit 52 for sensing and digitizing heart function data is shown in FIG. 5B.

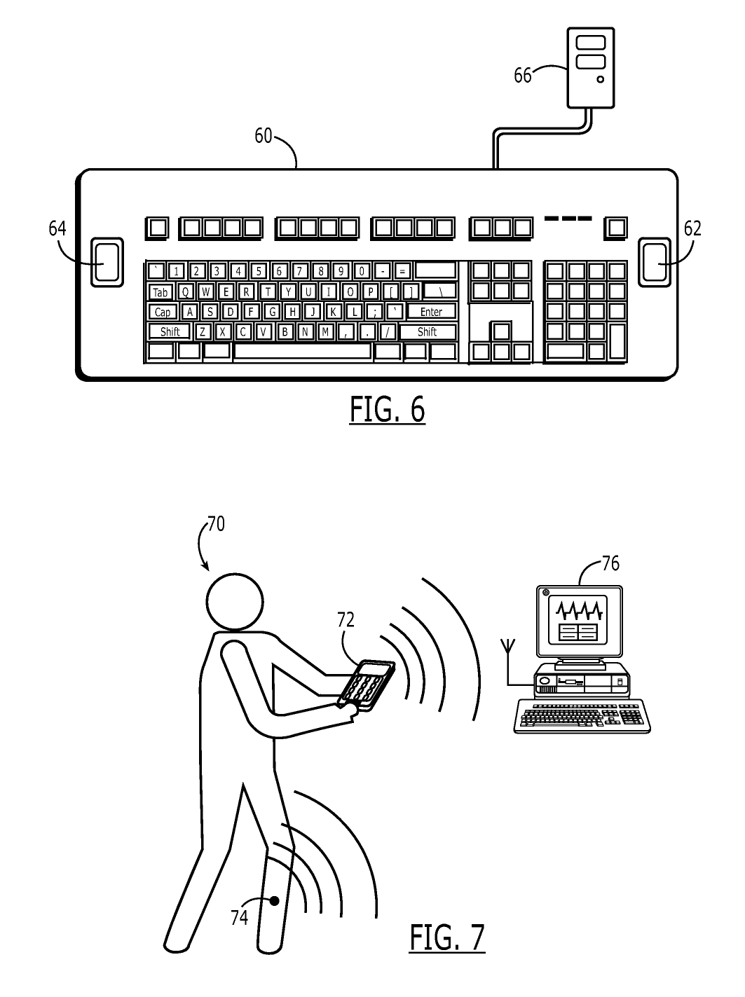

While a plurality of preferred exemplary embodiments have been presented in the foregoing detailed description, it should be understood that a vast number of variations exist, and these preferred exemplary embodiments are merely representative examples, and are not intended to limit the scope, applicability or configuration of the disclosure in any way. For example, while the description above illustrates the invention disclosed herein implemented in a cellular telephone handset, the invention may equally be integrated into many other different hand-held electronic devices such as personal digital assistant devices, digital music players, laptop and netbook computers, tablet computers and similar devices, remote control devices, and so forth. Furthermore, the present invention including a biometric sensor operable as both a sensor and an electrode in a heartbeat detection circuit may be a part of devices not traditionally held in a user's hand such as door locks, desktop computer keyboards and so forth which include biometric sensors. An example of a keyboard 60 including a combined sensor 62 and additional electrode 64 according to the description above is shown in FIG. 6. A user may use sensor 62 to log in to the operating system of a computer 66 to which keyboard 60 is attached. In addition, the user may place the finger of one hand on sensor 62 (for example an area sensor), and a finger of the other hand on electrode 64, and software operating on computer 66 may determine from data from sensor 62 and electrode 64 parametric data relating to a user's heart.

Still further, the description above has focused on discrete regions of the apparatus including a biometric sensor that operates as an electrode for the heartbeat monitoring circuit. However, in a variation of this description, the apparatus may be provided with a metal encasement that in its entirety may serve as a first electrode. That is, first electrode is not a discrete portion of the case of the apparatus, but rather is the entire case of the apparatus. Thus, a user may contact the case in any manner comfortable to the user, as opposed to requiring the user to hold the apparatus in a particular position or orientation.

And, while the above has focused on two electrodes (points of contact) within a single case, in certain embodiments, additional electrodes may be provided external to the case. FIG. 7 illustrates a user 70 holding a portable device 72 of the type described above providing two electrodes. A third electrode 74 may be provided, for example secured to the leg of user 70. Third electrode 74 may be communicatively connected to device 72 by wire or wirelessly. All data from electrodes forming part of device 72 and electrode 74 may be processed within device 72. Alternatively, all data from electrodes forming part of device 72 and electrode 74 may be transmitted to an optional processing center 76, by wired connection or wirelessly, for processing at that center. Other variations include device 72 and third electrode 74 wirelessly communicating with optional processing center 76 that receives data from device 72 and electrode 74 for analysis and determination of parametric data relating to the user's heart.

Finally, various presently unforeseen or unanticipated alternatives, modifications variations, or improvements therein or thereon may be subsequently made by those skilled in the art which are also intended to be encompassed by the claims, below.

Therefore, the foregoing description provides those of ordinary skill in the art with a convenient guide for implementation of the disclosure, and contemplates that various changes in the functions and arrangements of the described embodiments may be made without departing from the spirit and scope of the disclosure defined by the claims thereto.

What is claimed is:

1. An apparatus for physical and electrical connection with a user's body for monitoring electrical cell function parameters, comprising:
a first electrode for connecting with a first portion of the user's body;
a second electrode for connecting with a second portion of the user's body, said second electrode associated with a fingerprint sensor;
circuitry for processing electrical signals obtained from said first and second electrodes to produce parametric data relating to said user's electrical cell function.

2. The apparatus of claim 1, further comprising circuitry for analyzing data obtained from said fingerprint sensor about a fingerprint of the user, wherein said circuitry to produce parametric data relating to a user's electrical cell function forms a part of the circuitry for analyzing data obtained from said fingerprint sensor about the fingerprint of the user.

3. The apparatus of claim 1, wherein said apparatus includes a case having a metallic portion, and said first electrode is a region of said metallic portion of said case.

4. The apparatus of claim 1, wherein said second electrode forms a functional part of said fingerprint sensor.

5. The apparatus of claim 4, wherein said apparatus is a hand-held device including a processor capable of running various discrete application software packages, and said circuitry for processing electrical signals obtained from said first and second electrodes is operated under the control of an electrical cell function monitoring application capable of running on said processor.

6. The apparatus of claim 5, wherein said second electrode operates in a first mode as a component of a fingerprint sensor and in a second mode as an electrode of an electrical cell function monitor.

7. The apparatus of claim 6, wherein said electrical cell function monitoring application controls the switching of the operation of said second electrode between said first mode and said second mode.

8. The apparatus of claim 4, further comprising circuitry for analyzing data obtained from said fingerprint sensor about a fingerprint of the user, wherein said second electrode operates in a first mode as a component of a fingerprint sensor and in a second mode as an electrode of an electrical cell function monitor, and further wherein said circuitry for analyzing data obtained from said fingerprint sensor about a fingerprint of the user controls the switching of the operation of said second electrode between said first mode and said second mode.

9. The apparatus of claim 1, wherein said apparatus collects and determines parametric data relating to the user's heart activity, and further wherein said first portion of a user's body is on a first side of said user's body, said second portion is on a second side of said user's body opposite the first side of the user's body relative to the user's heart.

10. The apparatus of claim 1, further comprising a third electrode communicatively coupled to said apparatus, said third electrode for connecting with a third portion of a user's body in order to provide electrical cell function data for said third portion of said user's body to said apparatus.

11. The apparatus of claim 10, wherein said first electrode is for connecting with a first finger on a first hand of the user, said second electrode is for connecting with a first finger on a second hand of the user, and said third electrode is for connecting with a third portion of the user's body other than said first fingers of said first and second hands of the user.

12. The apparatus of claim 11, wherein said third portion of the user's body is other than a portion of said first and second hands.

13. The apparatus of claim 1, wherein said apparatus is selected from the group consisting of: cellular telephones, personal digital assistant devices, portably music players, laptop computers, netbook computers, tablet computers, and remote control devices.

14. The apparatus of claim 1, wherein said apparatus is communicatively connected to a processing center, and wherein said electrical signals obtained from said first and second electrodes are sent wirelessly to said processing center for analysis and determination of parametric data relating to said user's electrical cell functions.

15. A hand-held apparatus including biometric sensing and heart function monitoring, comprising:
a case;
a device housed within said case, at least certain functions performed by said device being other than fingerprint sensing, identification, and verification;
a fingerprint sensor disposed on one surface of said case;
circuitry for receiving signals from said fingerprint sensor and for creating a digitized representation of a user's fingerprint therefrom;
a first electrode disposed on said case for connecting with a first portion of the user's body;
a second electrode, forming a portion of said fingerprint sensor, for connecting with a second portion of the user's body; and
circuitry for processing electrical signals obtained from said first and second electrodes to produce parametric data relating to said user's heart.

16. The apparatus of claim 15, wherein said first portion of a user's body is on a first side of said user's body, and said second portion is on a second side of said user's body opposite the first side of the user's body relative to the user's heart.

17. The apparatus of claim 15, wherein said circuitry for processing electrical signals obtained from said first and second electrodes forms a part of the circuitry for receiving signals from said fingerprint sensor and for creating a digitized representation of a user's fingerprint.

18. The apparatus of claim 15, wherein said apparatus is selected from the group consisting of: cellular telephones, personal digital assistant devices, portably music players, laptop computers, netbook computers, tablet computers, and remote control devices.

19. The apparatus of claim 15, wherein said apparatus is a cellular telephone including a processor capable of running various discrete application software packages, and said circuitry for processing electrical signals obtained from said first and second electrodes is operated under the control of a heart monitoring application capable of running on said processor.

20. The apparatus of claim 19, wherein said fingerprint sensor includes a conductive region which operates in a first mode as a portion of a fingerprint sensor and in a second mode as said second electrode, and further wherein said heart monitoring application controls the switching of the operation of said conductive region between said first mode and said second mode.

21. The apparatus of claim 19, further comprising circuitry for analyzing data obtained from said fingerprint sensor about a fingerprint of the user, wherein said second electrode operates in a first mode as a component of a fingerprint sensor and in a second mode as an electrode of a parametric heart data monitor circuit, and further wherein said circuitry for analyzing data obtained from said fingerprint sensor about a fingerprint of the user controls the switching of the operation of second electrode between said first mode and said second mode.

22. The apparatus of claim 15, further comprising a third electrode communicatively coupled to said apparatus, said third electrode for connecting with a third portion of a user's body in order to provide electrical cell function data for said third portion of said user's body to said apparatus.

23. The apparatus of claim 22, wherein said first electrode is for connecting with a first finger on a first hand of the user, said second electrode is for connecting with a first finger on a second hand of the user, and said third electrode is for connecting with a third portion of the user's body other than said first and second hands of the user.

24. The apparatus of claim 15, wherein at least certain operations of said device housed in said case require authentication of a user by way of determining a match between the user's fingerprint and one or more pre-enrolled fingerprints.
